(19) United States
(12) Patent Application Publication
(10) Pub. No.: US 2023/0338472 A1
(43) Pub. Date: Oct. 26, 2023

(54) METHOD OF ENCAPSULATION OF AN ACTIVE PROTEIN USING ELECTRODEPOSITION TECHNIQUES, AN IMMUNOMODULATING COMPOSITION CONTAINING THE ACTIVE PROTEIN AND A POLYMER, AND ITS USE FOR THE PRODUCTION OF A PHARMACEUTICAL COMPOSITION FOR THE TREATMENT OF ATOPIC DERMATITIS IN HUMANS

(71) Applicant: BIOCELTIX SPOLKA AKCYJNA, Wroclaw (PL)

(72) Inventors: Lukasz BZDZION, Wroclaw (PL); Jakub GRZESIAK, Wroclaw (PL); Karol WRZESZCZ, Wroclaw (PL)

(21) Appl. No.: 18/005,051

(22) PCT Filed: Jul. 8, 2021
(86) PCT No.: PCT/IB21/56123

§ 371 Date: Jan. 10, 2023

(30) Foreign Application Priority Data
Jul. 10, 2020 (PL) 434621

(51) Int. Cl. A61K 38/19; A61K 35/28; A61K 47/10; A61K 47/32; A61P 37/02; A61P 17/00; C12N 5/0775; D01F 4/00; D01F 1/10

(52) U.S. Cl. CPC A61K 38/195; A61K 35/28; A61K 47/10; A61K 47/32; A61P 37/02; A61P 17/00; C12N 5/0662; D01F 4/00; D01F 1/10; D10B 2509/00

(57) ABSTRACT

The method of encapsulating an active protein includes (a) establishing a primary mesenchymal cell culture; (b) maintaining the cell culture established until the culture surface is fully covered by the cultured cells; (c) obtaining a culture fluid from the cultured cells; (d) purifying the culture fluid from cell debris and suspended cells; (e) transferring the upper liquid phase to a new vessel; (f) gently mixing the purified liquid phase with an aqueous solution of polyvinyl alcohol; (g) adding ethyl alcohol to the mixture while stirring continuously; and (h) depositing on the collector surface by electro spinning or electrospraying. The invention includes an immunomodulating composition containing an active protein and a polymer, wherein the active protein is a fibrous, fully water-soluble material containing proteins released by mesenchymal cells, including CCL2.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### Field of the Invention

The invention relates to a method of encapsulation of an active protein using electrodeposition techniques, an immunomodulating composition containing the active protein and a polymer, and its use for the production of a pharmaceutical composition for the treatment of atopic dermatitis in humans.

### 2. Description of Related Art Including Information Disclosed Under 37 CFR 1.97 and 37 CFR 1.98

The techniques of electrospinning and electrospraying are widely used in tissue engineering.

The solution known from the European patent application EP2254608 A2 describes a method that uses cell extracts to create scaffolds for tissue regeneration, as well as the application of cells to redesign the scaffolds in order to obtain the desired features. The disclosed method comprises: (a) Obtaining cells or tissues; (b) Preparing extracellular extracts and/or intracellular extracts from said cells or tissues; (c) Preparing a scaffold from said extracellular and/or intracellular extracts (preferably by electrospinning); (d) Redesigning said scaffolds by seeding cells thereon; (e) Eliminating the cells from the scaffold; and solubilising the scaffold, thereby obtaining an injectable scaffold formulation. Preferably, said intracellular extracts are prepared from separate cellular compartments, selected from a group consisting of a cytosolic compartment, a cytoplasmic compartment, a nuclear compartment, and any combination thereof.

The solution known from the international patent application WO2008039530 A2 relates to tissue engineering and includes an engineered intervertebral disc, comprising a nanofibrous polymer support comprising one or more polymer nanofibres; a hydrogel composition comprising at least one or more hydrogel materials; and a plurality of cells which are dispersed throughout the tissue engineered intervertebral disc, Wherein the nanofibrous polymer support preferably comprises poly(glycolide) (PGA), poly(L-lactic acid) (PLA), poly(lactide-co-glycolide) (PLGA), poly(L-lactide) (PLLA), poly(D,L-lactide) (P(DLLA)), polyethylene glycol (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNm Ph), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(3-hydroxybutyrate-co-3-hydroxyvalerate), poly(ethylene-co-vinyl alcohol), and combinations thereof.

The literature shows that electrospun fibres of different polymers with different diameters and/or morphology have already been tested as media for MSC cultivation. These scaffolds reveal significant biocompatibility with MSCs, which promotes their adhesion and growth in in cultures. In addition, recent research has demonstrated the overall flexibility of fibre scaffolds to support MSC differentiation (Braghirolli D. I., Steffanes D., Pranke P. Electrospinning for regenerative medicine: a review of the main topics. Drug Discovery Today 2014, http://dx.doi.org/10.1016/j.drudis.2014.03.024).

So far, various types of biomaterials have been used to produce electrospun scaffolds for the treatment of skin defects and burns. Electrospun scaffolds can support the adhesion and proliferation of fibroblasts and keratinocytes, and can also support MSC growth and differentiation into epidermal lineage cells. Electrospun fibres can also be combined with angiogenic and/or vascular factors, epidermal factors, and molecules with anti-inflammatory and antimicrobial properties to promote and improve skin regeneration. The nanofibre scaffolds constitute a good substrate for the adhesion and proliferation of MSCs and have appropriate physicochemical properties for use as skin substitutes (Braghirolli D. I., Steffanes D., Pranke P. Electrospinning for regenerative medicine: a review of the main topics. Drug Discovery Today 2014, http://dx.doi.org/10.1016/j.drudis.2014.03.024).

On the other hand, the publication by Hashizume et al. describes the use of the wet electrospinning technique to create polymer scaffolds, using poly(ester urethane)urea (PEUU) and DMEM (Dulbecco's Modified Eagle Medium, Invitrogen) cell culture medium supplemented with serum and antibiotics, wherein the DMEM medium was administered with an infusion pump at a rate of 0.2 ml/min into a sterilised capillary charged at 7 kV and suspended 4 cm above the target spindle. Simultaneously, PEUU in hexafluoroisopropanol solution (12%, w/v) was administered from a capillary at 1.5 ml/h, charged at 12 kV and perpendicularly, 20 cm from the target spindle. The spindle was charged at 4 kV and rotated at 250 rpm (tangential speed 8 cm/s), moving back and forth 8 cm along the x axis at a speed of 0.15 cm/s (Hashizume R., Fujimoto K. L., Hong Y., Amoroso N.J., Tobita K., Miki T., Keller B. B., Sacks M. S., Wagner W. R. Morphological and mechanical characteristics of the reconstructed rat abdominal wall following use of a wet electrospun biodegradable polyurethane elastomer scaffold. Biomaterials 2010; 31: 3253-3265).

In addition, the available literature indicates that electrospinning and electrospraying techniques are used to encapsulate active ingredients such as growth factors, alpha-lipoinic acid, anti-inflammatory drugs (e.g. naproxen), contraceptives, hormonal drugs (Bock N., Dargaville T. R., Woodruff M. A. Electrospraying of polymers with therapeutic molecules: State of the art. Progress in Polymer Science 2012; 37: 1510-1551).

Adipose tissue derived mesenchymal stromal cells are characterised by a high immunomodulatory potential (Reza Abdi,1 Paolo Fiorina,1,2 Chaker N. Adra,1,3 Mark Atkinson,4 and Mohamed H. Sayegh1,3, Immunomodulation by Mesenchymal Stem Cells. A Potential Therapeutic Strategy for Type 1 Diabetes, Diabetes. 2008 July; 57(7): 1759-1767; Poggi A1, Zocchi MR2. Immunomodulatory Properties of Mesenchymal Stromal Cells: Still Unresolved “Yin and Yang”, Curr Stem Cell Res Ther. 2019; 14(4):344-350. doi: 10.2174/1574888X14666181205115452; A Gebler, 0 Zabel, B Seliger, The immunomodulatory capacity of mesenchymal stem cells, Trends in molecular medicine, 2012, Volume 18, Issue 2, February 2012, Pages 128-134). The substances with confirmed bioactivity against cells of the immune system include, among others CCL2 (MCP-1) and TGFβ (Rafei et al., Mesenchymal stromal cell—derived CCL2 suppresses plasma cell immunoglobulin production via STAT3 inactivation and PAXS induction, Blood (2008) 112 (13): 4991-4998; de Araujo Farias et al., TGF-β and mesenchymal stromal cells in regenerative medicine, autoimmunity and cancer, Cytokine Growth Factor Rev. 2018 October; 43:25-37. doi: 10.1016/j.cytogfr.2018.06.002). Numerous studies have shown the beneficial effect of substances secreted by mesenchymal cells on the symptoms of atopic dermatitis (Kim et al., Human Adipose Tissue-Derived Mesenchymal Stem Cells Attenuate Atopic Dermatitis by Regulating the Expression of MIP-2, miR-122a-SOCS1 Axis, and Th1/Th2 Responses, Front Pharmacol. 2018; 9: 1175; Park et al., TGF-β secreted by human umbilical cord blood-derived mesenchymal stem cells ameliorates atopic dermatitis by inhibiting secretion of TNF-α and IgE, Stem Cells. 2020 Apr. 11. doi: 10.1002/stem.3183).

These substances are released into the medium in the process of cell proliferation in in vitro culture and can be isolated in various ways. For example, from the publication by Si and Yang, the acid-alcohol method of TGF beta extraction is known (Si X-H., Yang L-J. Extraction and purification of TGFβ and its effect on the induction of apoptosis of hepatocytes. World J Gastroenterol 2001; 7(4) 527-531).

However, the above-mentioned substances produced by mesenchymal cells are of limited persistence due to their proteinaceous nature.

The study by Felice et al. indicate that with the use of the electrohydrodynamic synthesis process, it is possible to obtain micro- and nanofibres, or micro- and nanoparticles in a dry, water-soluble form, which, after dissolution, release undegraded and fully functional proteins and peptides. In the study, low-molecular-weight (20-30 kDa) and high-molecular-weight (89-124 kDa) polyvinyl alcohol was used. In order to ensure the ion content in the substrate was suitable for the synthesis process, the authors used glacial acetic acid. In order to obtain the surface tension and viscosity of the substrate suitable for the process, the authors used anhydrous ethyl alcohol. The study of the above-mentioned authors showed that it is possible to obtain water-soluble fibres or particles containing fully functional insulin (Felice B., Prabhakaran M. P., Zamani M., Rodriguez A. P., Ramakrishna S. Electrosprayed poly(vinyl alcohol) particles: preparation and evaluation of their drug release profile. Polym Int. 2015; 64:1722-1732).

### BRIEF SUMMARY OF THE INVENTION

The object of the invention is to provide a process for obtaining a mesenchyme-derived active protein with an extended durability.

The subject of the invention is a method of encapsulating an active protein using electrodeposition techniques, characterised in that it comprises the following steps:
establishing a primary mesenchymal cell culture containing 2,000-5,000 source tissue cells and a serum-supplemented culture medium;
maintaining the cell culture established in step (a) for 280-340 hours until the culture surface is fully covered by the cultured cells;
obtaining a culture fluid from the above of the cultured cells;
purifying the culture fluid obtained in step (c) from cell debris and suspended cells by centrifuging said fluid with a force of 300 to 1200×g;
transferring the upper liquid phase from above the sediment to a new vessel;
gently mixing the purified liquid phase obtained in step (e) with an aqueous solution of polyvinyl alcohol;
adding ethyl alcohol to the mixture obtained in step (f) while stirring continuously;
the material obtained in step (g) is deposited on the collector surface by means of electrospinning or electrospraying.

Preferably the method comprises step (e′), wherein the liquid phase purified from the cells is further purified from proteins greater than 50 kDa by filtering.

Preferably, establishing the culture in step (a) is performed using a culture medium selected from the group consisting of DMEM (Dulbecco Modified Eagle Medium), DMEM-Ham's F-12 (Dulbecco Modified Eagle Medium-Ham's F-12), IMDM (Iscove's Modified Dulbecco Medium).

Preferably, the mesenchymal cells used in step a) are mesenchymal stromal cells derived from adipose tissue, bone marrow or Wharton's jelly.

Preferably, the mesenchymal cells are mesenchymal cells of species selected from the group consisting of dogs, cats, horses and sheep.

Another subject of the invention is an immunomodulating composition containing an active protein and a polymer, characterised in that it contains ethyl alcohol, wherein the active protein is a fibrous, fully water-soluble material containing proteins released by mesenchymal cells, including CCL2 at an amount from 0.56 to 5.62 ng/g of dry weight of the composition, and the polymer is an aqueous solution of polyvinyl alcohol.

Preferably, the polymer is a 30% aqueous solution (300 mg/ml) of polyvinyl alcohol.

Preferably, the composition comprises 47.5% of the active protein, 47.5% of polyvinyl alcohol aqueous solution and 5% of ethyl alcohol.

Preferably, the mesenchymal cells are mesenchymal stromal cells derived from adipose tissue, bone marrow or Wharton's jelly.

Preferably, the mesenchymal cells are mesenchymal cells of species selected from the group consisting of dogs, cats, horses and sheep.

Another subject of the invention is the use of a composition according to the invention for the preparation of a pharmaceutical composition for the treatment of atopic dermatitis in humans.

The invention provides the following advantages:
extended durability of the formulation according to the invention;
immunomodulatory effect—inhibiting the activation of leukocytes;
the effect of reducing the level of antibodies in the course of atopic dermatitis;
simplification and reduction of costs of processing the post-culture media containing the active ingredient as compared to traditional methods of recovering proteins from post-culture fluids.

### BRIEF DESCRIPTION OF THE SEVERAL VIEWS OF THE DRAWINGS

The invention is shown in the Drawings.

FIG. 1 is a series of electron microscope images, representing the micro- and ultrastructure of the product obtained by the method according to the invention visualised using a scanning electron microscope under the following magnifications: A) 250×, B) 500×, C) 6500×, D) 35000×.

FIG. 2 is a graph illustration, representing the cytotoxicity assay for the compositions of the invention against three cell populations—or lines D-17, HAP1 and MSC.

FIG. 3 are graph illustrations, presenting the effect of the invention on the activation level of mitogen-stimulated murine splenocytes under in vitro conditions.

FIG. 4 is a graph illustration, presenting the effect of the use of the invention on the level of IgE antibodies in animals with experimentally induced atopic dermatitis.

FIG. 5 is a graph illustration, presenting the CCL2 concentration change in the dry weight of the composition according to the invention during refrigerated storage.

FIG. 6 is a graph illustration, showing the change in CCL2 concentration in the material obtained after step e′ of the method of the invention (or before electrodeposition) during refrigerated storage.

### DETAILED DESCRIPTION OF THE INVENTION

The invention is presented in details in the following embodiments, wherein all the tests and experimental procedures described below were performed using commercially available test kits, reagents and equipment, following the instructions of the manufacturers of the applied kits, reagents and equipment, unless expressly stated otherwise. All test parameters were measured using standard, commonly known methods used in the art to which this invention belongs. In vivo tests were carried out at the University of Life Sciences after obtaining the consent of the Ethics Committee to conduct an experiment on mice.

### Example 1

Method of Encapsulating an Active Protein

The method of encapsulating an active protein of the invention comprises the following steps:

Step a: Establishing a Primary Mesenchymal Cell Culture

The first step of the method according to the invention comprises establishing a primary mesenchymal cell culture containing (at the initial step) 2,000-5,000 source tissue cells (in a culture vessel) and a serum-supplemented culture medium (preferably 10% bovine serum). The culture is then maintained without the use of antibiotics. In the present embodiment, 5,000 cells were used to establish the culture. Cells were counted in the Bürker chamber,
wherein, the term “primary culture” is understood to mean a non-passaged culture, or a so-called “passage 0” obtained directly from a frozen tissue isolate constituting “stock culture”, or from direct inoculation of the tissue isolate and not subject to further culture passages.

In this non-limiting embodiment, canine bone marrow mesenchymal cells and DMEM (Dulbecco Modified Eagle Medium) supplemented with 10% serum were used. On the other hand, other mesenchymal cells (e.g., mesenchymal stromal cells derived from adipose tissue, bone marrow or Wharton's jelly isolated from species such as dog, cat, horse and sheep during in vitro culture) may also be used in the method of the invention. Similarly, in the case of the culture medium, to establish a culture medium of the above-mentioned mesenchymal cells, culture media containing ions necessary for the maintenance and growth of cells in culture other than DMEM, e.g. DMEM-Ham's F-12 (Dulbecco Modified Eagle Medium-Ham's F-12) or IMDM (Iscove's Modified Dulbecco Medium), which ensure the appropriate content of ions to obtain sufficient substrate conductivity for the proper performance of the material synthesis process in the electrodeposition process, can also be used,
wherein the choice of a specific medium depends on the mesenchymal cells selected for the culture. To obtain a complete culture medium, the medium is supplemented with 10% bovine serum. However, no antibiotics are added to the medium prepared in this way.

Stage b: Maintaining the Cell Culture Established in Step (a) Until the Culture Surface is Fully Covered by the Cultured Cells

Cell cultures are performed in standard culture vessels under conditions consistent with the guidelines of the American Type Culture Collection, ATTC), or at a temperature of 37° C. and an atmosphere containing 95% of air and 5% of CO2 at a relative humidity of 90%.

The culture is maintained under the above-mentioned conditions for 280 hours until the culture surface is fully covered by the cultured cells, wherein, during the culture process, the culture medium is not replaced. This will allow for the collection of all proteins, growth factors, cytokines and other peptides secreted by the mesenchymal cells from the beginning of the culture.

Step c: Obtaining a Culture Fluid from the Above of the Cultured Cells

After the mesenchymal cells cover the culture surface adequately, the culture fluid containing proteins, growth factors, cytokines and peptides secreted by the cultured cells is transferred into a new sterile vessel.

Step d: Purifying the Culture Fluid Obtained in Step (c) from Cell Debris and Suspended Cells.

The obtained culture medium is centrifuged at 1200×g in order to eliminate cell debris and suspended cells.

Step e: Transferring the Supernatant Liquid Phase to a New Vessel

After centrifugation, the obtained supernatant liquid phase is transferred to a new sterile vessel.

In this embodiment, the medium was then subjected to purification of albumin derived from foetal bovine serum, which may be an allergen. Filtration was performed using an Amicon-type molecular filter with a pore size of 50 kDa, thanks to which proteins larger than 50 kDa are removed from the said media, and proteins, growth factors, cytokines and peptides smaller than 50 kDa are preserved.

The media prepared in this way can be used to obtain the substrate directly on the day of their preparation, or they can be frozen at a temperature <−18° C. and used later after thawing.

Step f and g: Mixing the Purified Liquid Phase with an Aqueous Solution of Polyvinyl Alcohol and Ethyl Alcohol;

The next steps (f and g) include mixing the purified liquid phase with a solution of polyvinyl alcohol and ethyl alcohol. In this embodiment, the electrodeposition material is obtained by mixing the purified aqueous phase of the post-culture medium (component A) with a 30% polyvinyl alcohol solution (component B) and 99.8% ethyl alcohol (component C) at the following volume ratio:
47.5% of component A+47.5% of component B+5% of component C,
wherein, first, component A is mixed with component B in a gentle manner, preventing the formation of foam.

In this embodiment, a 30% aqueous solution (300 mg/ml) of polyvinyl alcohol with a molecular weight of 20-30 kDa is used as component B, wherein, the aqueous solution of polyvinyl alcohol is prepared by mixing 300 mg of dry polymer with 1 ml of water and heating the mixture at 90° C. until the polymer powder is completely dissolved (or approximately 1-2 hours). The solution is then cooled to room temperature.

After the components A and B are mixed, component C (ethyl alcohol) is added at continuous stirring. The material prepared in this way is used for electrodeposition, which should be started immediately in order to reduce the process of degradation of proteins present in the substrate.

Stage h: Electrodeposition of the Prepared Material on the Collector Surface

The prepared material (substrate) is loaded into a disposable syringe. The syringe is connected to a hose ending with a head that supplies electric voltage to the substrate, at the end of which a blunt tip steel needle is installed, wherein, in this embodiment, the outer needle diameter was 1 mm and the inner needle diameter was 0.7 mm.

A cable supplying positive voltage of 11.8 kV is connected to the head. The syringe is placed on an automatic piston connected to an adjustable stepper motor that allows the speed of liquid flow through the system to be set. The material is deposited on the surface of the collector, in particular a steel collector or an aluminium foil connected to the grounding. wherein the liquid flow rate is 60 μl of substrate/hour and the distance from the end of the needle to the collector is 12 cm, wherein, in this embodiment, the deposition of the material on the collector surface may be performed by electrospinning.

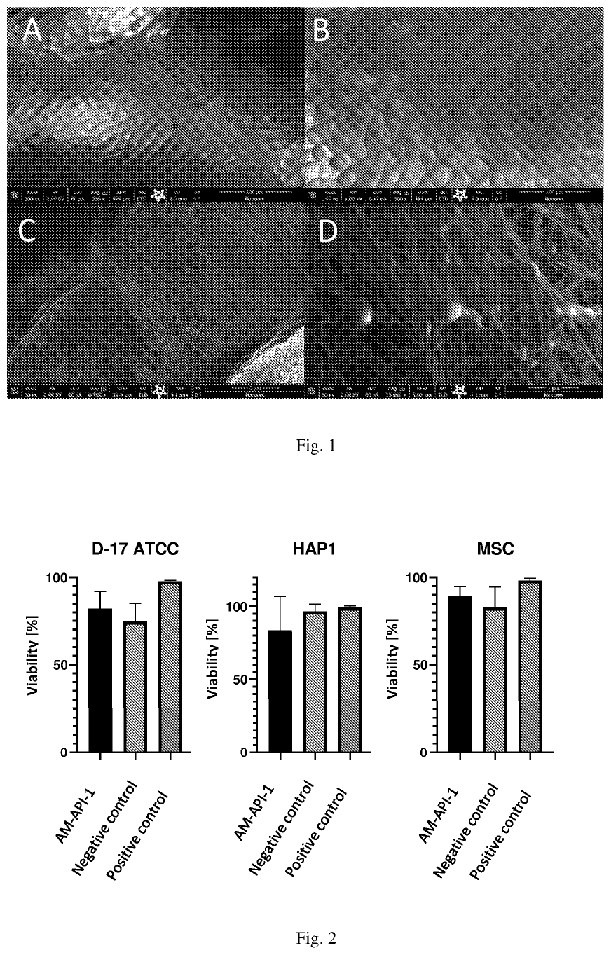

The process allows for the production of a minimum of 4 mg of dry product from 60 μl of substrate per hour (28.5 μl of culture medium), wherein the obtained product contains 6.5% of protein in dry matter. The micro- and ultrastructure of the obtained material are shown in FIG. 1.

### Example 2

Method of encapsulating an active protein according to Example 1, except that: human mesenchymal cells of bone marrow or Wharton's jelly and IMDM medium supplemented with 10% serum were used to establish the primary mesenchymal cell culture.
wherein 2,000 cells were used, and the culture was maintained for 340 hours. The obtained culture medium was centrifuged at 300×g in order to eliminate cell debris and suspended cells.

An aqueous solution of polyvinyl alcohol was prepared by mixing 300 mg of dry polymer with 1 ml of water and heating the mixture at 95° C. for 1 hour and then cooling to room temperature.

The electrodeposition substrate was loaded into a disposable syringe with the outer needle diameter of 0.5 mm and the inner needle diameter of 0.2 mm.

In turn, the electrodeposition was carried out using the following parameters:
positive voltage of 11.7 kV;
liquid flow rate of 60 μl of substrate/hour
distance from the tip of the needle to the collector of 13 cm.
wherein, in this embodiment, the deposition of the material on the collector surface was performed by electrospraying.

The process allows for a product containing 5.5% of protein in dry matter.

### Example 3

Immunomodulating Composition

The product obtained by the method according to the invention is a composition with immunomodulatory properties.

Said composition comprises an active protein, a polymer, and ethyl alcohol, wherein, the active protein is a fibrous, fully water-soluble material containing proteins released by mesenchymal cells (e.g., canine bone marrow mesenchymal cells), including CCL2. In this embodiment, the mesenchymal cells are canine bone marrow mesenchymal cells. On the other hand, other mesenchymal cells e.g., mesenchymal stromal cells derived from adipose tissue, bone marrow or Wharton's jelly of human origin or isolated from species such as dog, cat, horse and sheep during in vitro culture may also be used during in vitro culture.

In this embodiment, the composition of the invention comprises CCL2 at an amount of 5.62 ng/g of the dry weight of the composition. In turn, the polymer is an aqueous solution of polyvinyl alcohol, preferably a 30% aqueous solution (300 mg/ml) of polyvinyl alcohol with a molecular weight of 20-30 kDa.

In this embodiment, the antibacterial composition of the invention comprises 47.5% of the active protein, 47.5% of polyvinyl alcohol aqueous solution and 5% of ethyl alcohol.

### Example 4

Composition as in Example 3, except that it contains CCL2 at an amount of 0.56 ng/g of dry weight of the composition.

### Example 5

Composition as in Example 3, except that it contains CCL2 at an amount of 3.75 ng/g of dry weight of the composition.

### Example 6

Composition as in Example 3, except that it contains CCL2 at an amount of 1.43 ng/g of dry weight of the composition.

### Example 7

Analysis of the cytotoxicity of the compositions of the invention

The cytotoxicity assay was performed using canine adipose tissue-derived mesenchymal stromal cells (MSC, passage 3), dog osteosarcoma reference line (ATCC® D-17), and human leukaemia haploid line (HAP1, HorizonDiscovery). For the experiment, cells were grown in wells in a 24-well plate.

The following sample designations were used:
AM-API-1 test sample—a 10% solution of the composition according to the invention in DMEM (Dulbecco Modified Eagle Medium) containing 10% foetal bovine serum;
negative control—10% solution of the negative material in DMEM containing 10% of foetal bovine serum, where the negative material is fresh (or has never come into contact with cells) medium encapsulated according to the method of the invention;
positive control—DMEM containing 10% of foetal bovine serum

In the first stage of the study, the cultures of the MSC, D-17 and HAP1 cell lines were established and the cultures were maintained until the culture surface was fully covered. After the culture surface was fully covered, a 10% solution of AM-API-1 in DMEM containing 10% of foetal bovine serum was added to the test group cultures. A 10% solution of AM-API-1 analogous material was added to the negative control culture, wherein the culture medium was replaced with fresh medium. Complete medium (DMEM+10% of foetal bovine serum) was added to the positive control. After 24 hours of culture, cells were harvested with the use of trypsin and tested for viability using 0.4% trypan blue (staining dead cells) and a BioRad TC20 automated cell counter. The analysis was performed in ten replicates for each cell line,
wherein the culture density (number of cells/cm2) differed between individual lines, wherein in the case of:
the MSC line—the culture density at the time of harvest after the experiment was ˜1×105 cells/cm2, while 10×˜1×105 cells were counted in the experiment;
the D-17 line—the culture density at the time of harvest after the experiment was ˜1.8×105 cells/cm2, while 10×˜1.8×105 cells were counted in the experiment;
the HAP1 line—the culture density at the time of harvest after the experiment was 3.2×105 cells/cm2, while 10×˜3.2×105 cells were counted in the experiment.

The conducted analysis revealed that the 10% AM-API-1 solution showed a slight cytotoxicity in relation to the tested cells of the D-17, HAP1 and MSC lines (FIG. 2). No statistically significant difference between the experimental group and the negative control was recorded, which indicates that polyvinyl alcohol is probably responsible for the slight cytotoxic effect, which may affect the properties of the medium (osmotic concentration, density).

### Example 8

Analysis of Immunomodulatory Properties Under In Vitro Conditions

The study of immunomodulatory properties was performed using murine splenocytes isolated according to the method previously described in the literature (Jun Feng Lim, Heidi Berger, I-hsin Su, Isolation and Activation of Murine Lymphocytes, J Vis Exp. 2016; (116): 54596). C57BI/6 murine splenocytes at an amount of 0.5×106 cells/ml in a 24-well plate (1 ml of medium/well) were cultured in RPMI medium supplemented with 10% foetal bovine serum (FBS) and in the presence of 20 mM of L-Gln and a cocktail of antibiotics and antifungal agents (Anti-Anti, Sigma Aldrich) in a CO2 incubator (5%). Cells were treated with a concanavalin A solution (Sigma Aldrich) at a concentration of 50 μg/ml with a 3% solution of AM-API-1 (or composition according to the invention) or a 3% solution of control material for 18 hours,

wherein the control material (or negative control) is fresh DMEM (or DMEM that has never come into contact with mesenchymal cells) containing 10% of bovine foetal serum, encapsulated according to the invention and dissolved at a concentration of 3% (w/v) in the medium used for the cultivation of splenocytes.

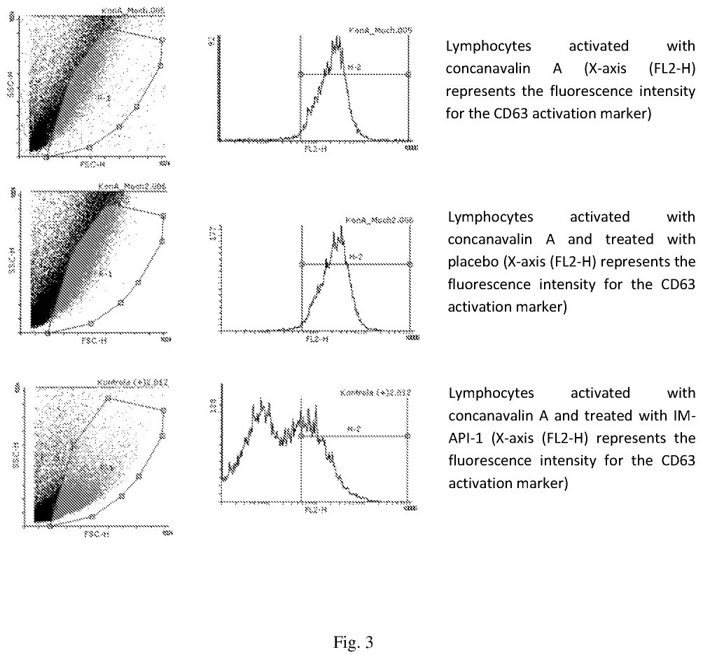

Cells were washed with PBS+2% FBS solution and stained with anti-CD69 PE antibody conjugate (clone #H1.2F3, 1/200). Cells were analysed using a BD FACS Calibur flow cytometer. The study showed that the presence of AM-API-1 (or the composition of the invention) in the medium significantly inhibited the activation and proliferation of murine splenocytes, which indicates the immunomodulatory effect of AM-API-1 (FIG. 3).

### Example 9

Analysis of Immunomodulatory Properties Under In Vivo Conditions

The study of immunomodulatory properties in an animal model was carried out using the model described previously (Kitamura et al. SCIeNTIFIC REporTs|(2018) 8:5988|DOI:10.1038/s41598-018-24363-6). Atopic skin lesions were induced in experimental animals by a double application (with a pause of 14 days) of a 0.15% solution of dinitrofluorobenzene (DNFB) in acetone and olive oil to freshly shaved and mechanically irritated dorsal skin (1 cm2). After the second application of the sensitising substance for 5 consecutive days (once a day), 20% solutions of AM-API-1 (or the composition according to the invention) or control material, respectively, were applied to the skin of the mice in the experimental and control groups. 10 mice/group were used,
wherein the control material (or negative control) is fresh DMEM (or DMEM that has never come into contact with mesenchymal cells) containing 10% of bovine foetal serum, encapsulated according to the invention and dissolved at a concentration of 20% in a sterile saline solution.

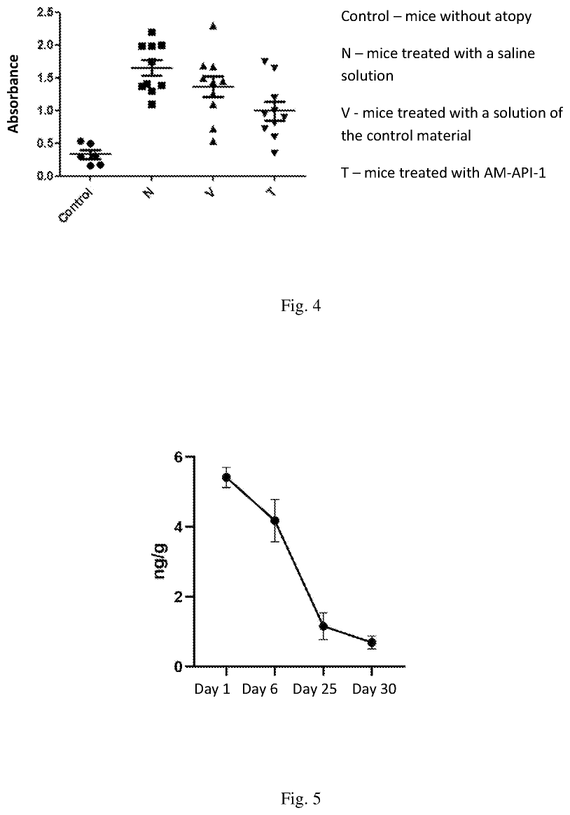

The solutions were prepared each day just before application by dissolving AM-API-1/control material in a sterile saline solution to a concentration of approximately 20% (w/v). On day 6, the mice were euthanised. Blood serum from all mice was tested in duplicate for IgE concentration using the mouse IgE Elisa set (BD OptEIA™) according to the manufacturer's instructions. The results showed that the use of the composition according to the invention significantly reduced the level of IgE in the mice from the experimental group. At the same time, animals from the control group (treated with a solution of control material) also showed a decreased level of serum IgE, which may indicate a beneficial effect of the medium and proteins derived from bovine foetal serum (FIG. 4).

The obtained results confirm the immunomodulatory properties of the compositions according to the invention, which in combination with the lack of cytotoxicity make the composition according to the invention suitable to be used for the production of medicinal products for the treatment of atopic dermatitis.

### Example 10

Analysis of the CCL2 Content According to the Invention

The basis for obtaining reproducible amounts of CCL2 in the composition according to the invention is obtaining conditioned media from cultures conducted according to a specific, reproducible and validated method, or the method according to the invention.

In this embodiment, the composition of the invention (designated AM-API-1) was obtained using mesenchymal cells derived from a dog.

The immunomodulatory properties of CCL2 (MCP-1) are well known from the professional literature (Bridgette D Semple, Tony Frugier & M Cristina Morganti-Kossmann; CCL2 modulates cytokine production in cultured mouse astrocytes, Journal of Neuroinflammation volume 7, Article number: 67 (2010); Derek S. Whelan, Noel M. Caplice & Anthony J. P. Clover, Mesenchymal stromal cell derived CCL2 is required for accelerated wound healing, Scientific Reports volume 10, Article number: 2642 (2020)).

A commercially available ELISA (Canine CCL2/MCP-1 Quantikine ELISA Kit, R&D Systems) was used to confirm the CCL2 (MCP-1, hereinafter CCL2) content in the composition of the invention (hereinafter AM-API-1).

In order to perform a measurement, a 10% solution of AM-API-1 was prepared by dissolving 100 mg of the material (composition according to the invention) in 1 ml of DMEM. As a negative control, the material prepared according to the invention (analogous to AM-API-1) was applied, which was prepared using fresh DMEM containing 10% of serum instead of conditioned medium obtained from the culture (or fresh, sterile medium that had never come into contact with tissue culture was used). The assay was prepared according to the instructions provided by the manufacturer with the ELISA kit (Canine CCL2/MCP-1 Quantikine ELISA Kit, R&D Systems). Measurements were performed using a ThermoScientific Multiskan FC spectrophotometric microplate reader.

The study was carried out in five replicates with the use of five different batches of the culture medium. In each replicate, the control and test sample were prepared using five independent source cell cultures.

In the study, both the peptide content in the dry weight of the AM-API-1 composition on the first day after isolation, and the stability of the material stored under refrigerated conditions (2-8° C.) over a 30-day period were determined. The results are shown in Table 1 below:

TABLE 1
Analysis of CCL2 content in dry matter of AM-API
formulation during 30 days of refrigerated storage

| Time (days) | 1 | 6 | 25 | 30 |
|---|---|---|---|---|
| Content in 1 g | 5.21-5.62 | 3.75-4.61 | 0.89-1.43 | 0.56-0.82 |
| of dry matter | | | | |
| (ng) | | | | |

It has been shown that refrigerated storage allows the stability of the test protein in AM-API-1 to be maintained for a minimum of 30 days (FIG. 5).

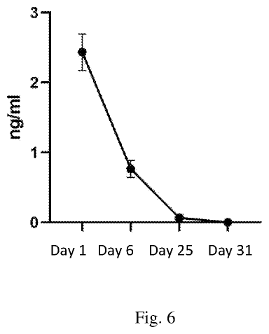

Moreover, the concentration of CCL2 in the conditioned media constituting the starting material for AM-API-1 (or the material obtained after step e′ of the method according to the invention, but before its mixing with the aqueous solution of polyvinyl alcohol) was tested analogously. The media were stored under refrigerated conditions (or at a temperature in the range of 2-8° C.) for no more than 30 days after being harvested from the culture. The media were stored in the refrigerator for 10 consecutive days to determine the decrease in peptide concentration over time. Concentration was measured on days 1, 6, 25 and 30 after the collection of the culture media. The test was performed in triplicate. CCL2 concentration was measured using an ELISA assay (Canine CCL2/MCP-1 Quantikine ELISA Kit, R&D Systems). As shown in FIG. 6, after the collection of the culture medium, the CCL2 concentration was above 2 ng/ml, after 6 days of refrigerated storage of the medium, the CCL2 concentration dropped to a level below 1 ng/ml, on day 25 CCL2 was detectable in some samples, while after 30 days CCL2 was not was detectable in any of the samples.

The presented analysis confirms that the encapsulation of the active ingredient using the method according to the invention provides a product with extended stability.

1. A method of encapsulating an active protein using electrodeposition techniques, the method comprising the following steps:
(a) establishing a primary mesenchymal cell culture containing 2,000-5,000 source tissue cells and a serum-supplemented culture medium;
(b) maintaining the cell culture established in step (a) for 280-340 hours until the culture surface is fully covered by the cultured cells;
(c) obtaining a culture fluid from the above of the cultured cells;
(d) purifying the culture fluid obtained in step (c) from cell debris and suspended cells by centrifuging said fluid with a force of 300 to 1200×g;
(e) transferring the upper liquid phase from above the sediment to a new vessel;
(f) gently mixing the purified liquid phase obtained in step (e) with an aqueous solution of polyvinyl alcohol;
(g) adding ethyl alcohol to the mixture obtained in step (f) while stirring continuously; and
(h) depositing the material obtained in step (g) on the collector surface by means of electrospinning or electrospraying.

2. The method, according to claim 1, wherein establishing the culture in step (a) is performed using a culture medium selected from the group consisting of DMEM, DMEM-Ham's F-12, IMDM.

3. The method, according to claim 1, wherein the mesenchymal cells used in step a) are mesenchymal stromal cells derived from adipose tissue, bone marrow or Wharton's jelly.

4. The method, according to claim 3, wherein the mesenchymal cells are mesenchymal cells of species selected from the group consisting of dogs, cats, horses and sheep.

5. An immunomodulating composition, comprising:
an active protein;
a polymer; and
ethyl alcohol,
wherein the active protein is a fibrous, fully water-soluble material containing proteins released by mesenchymal cells, including CCL2 at an amount from 0.56 to 5.62 ng/g of dry weight of the composition, and the polymer is an aqueous solution of polyvinyl alcohol.

6. The immunomodulating composition, according to claim 5, wherein the polymer is a 30% aqueous solution (300 mg/ml) of polyvinyl alcohol.

7. The immunomodulating composition, according to claim 5, wherein the mesenchymal cells are mesenchymal stromal cells derived from adipose tissue, bone marrow or Wharton's jelly.

8. The immunomodulating composition, according to claim 7, wherein the mesenchymal cells are mesenchymal cells of species selected from the group consisting of dogs, cats, horses and sheep.

9. A method of using a composition, the method comprising the step of:
preparing the composition, according to claim 5 as a pharmaceutical composition; and
treating atopic dermatitis in humans.
